(19) United States
(12) Patent Application Publication
(10) Pub. No.: US 2021/0151142 A1
(43) Pub. Date: May 20, 2021

(54) IMPLEMENTATION OF AN ANIMAL BLOCKCHAIN USING UNIQUE ID FOR ANIMAL IDENTITY AND RECORDS MANAGEMENT

(71) Applicants: Stephanie Sujin CHOI, Seoul (KR); Hyeong In CHOI, Seoul (KR)

(72) Inventors: Stephanie Sujin CHOI, Seoul (KR); Hyeong In CHOI, Seoul (KR)

(21) Appl. No.: 17/098,756

(22) Filed: Nov. 16, 2020

Related U.S. Application Data
(60) Provisional application No. 62/935,740, filed on Nov. 15, 2019.

(51) Int. Cl. G16H 10/60; G06Q 10/10; G06Q 30/00; G16H 40/20; G06Q 40/04; G06Q 20/38; G06Q 20/06; G06F 16/23; G06F 21/62; G06K 9/00

(52) U.S. Cl. CPC G16H 10/60; G06Q 10/10; G06Q 30/0185; G16H 40/20; G06Q 40/04; G06Q 50/26; G06Q 20/0655; G06F 16/2379; G06F 21/6218; G06K 9/00362; G06Q 2220/00; G06Q 20/389

(57) ABSTRACT

An animal blockchain for the enrollment of individual animals using unique ID and management of all relevant data and records pertaining to each enrolled animal. The animal blockchain has as its features a separation of animal ID from the owner ID by assigning unique identifiers to the animals, categorical separation of the animal registration data from the human registration data, keeping of animal records linked to each unique animal ID, as well as use of an external storage to minimize the size of each block.

### CROSS REFERENCE TO RELATED APPLICATION

This application claims the benefit of U.S. Provisional Application No. 62/935,740, filed Nov. 15, 2019, the disclosure of which is incorporated by reference in its entirety herein.

### BACKGROUND OF THE INVENTION

### 1. Field of the Invention

The present invention relates to an animal blockchain for the enrollment of individual animals using unique ID and management of all relevant data and records pertaining to each animal.

### 2. Description of Related Art

Companion animals, especially dogs and cats, have over the years solidified their place in human society as family members. On the other hand, when it comes down to their station in human society, they are still treated as ‘things’, a part of the mundane production, distribution, possession, trade, and disposition cycle as any other product. As a ‘thing,’ a companion animal does not have its own identity. Its identity or even its existence is acknowledged only in relation to its owner. To appreciate such a predicament, one needs only to think of the case of a lost or stray dog in which all medical history data, including vaccination records, are lost.

### SUMMARY OF THE INVENTION

In one general aspect, there is provided an animal blockchain including an enrollment of animals onto the blockchain using a unique Animal ID (AID).

In another general aspect, there is provided an animal blockchain including a unique Animal ID (AID) and Guardian ID (GID) as paired entities.

### BRIEF DESCRIPTION OF THE DRAWINGS

FIG. 1 illustrates data storage and block generation at the time of enrollment.

FIG. 2 illustrates inputting an Animal Record onto the blockchain.

### DETAILED DESCRIPTION OF THE INVENTION

The preferred embodiment of the present invention pertains to certain structural features as the separation of animal ID from the owner ID by assigning unique identifiers to the animals, categorical separation of the animal registration data from the human registration data, keeping of animal records linked to each unique animal ID, as well as the use of an external storage to minimize the size of each block. However, there are many blockchain design elements that are not explicitly described here to which the present invention's spirit and methodology can be easily applied to achieve the same goal of ID and records management for individual animals on a blockchain. Therefore, the scope of the present invention should be interpreted as covering such omissions and variations. Moreover, the preferred embodiment given here is only one example of how the present invention can be applied to the general problem of implementing an animal ID-based blockchain and many similar variations are possible without going beyond the scope of the present invention.

The present invention thereby addresses two fundamental issues: first, how to endow to each animal a unique identity that is independent of its owner; and second, how to keep each animal's record in an immutable public ledger as belonging to the animal itself, instead of the owner, so that the record is always available to all concerned public even if its owner cannot be ascertained.

The present invention solves the above two issues by demonstrating an animal blockchain onto which animals are enrolled using a unique Animal ID (AID), under which Animal Records are created, managed, put on the blockchain, and made accessible to authorized third parties, the general public, or whoever has access privilege granted by the design of the blockchain. In the blockchain, the AID is linked to the animal's intrinsic biometric data, which may be conversely used to retrieve the AID through the biometric identification of an animal. The AID of an animal may be paired with a Guardian ID (GID) if an owner of the animal exists, where the pairing may change during the animal's lifetime and the AID may be used to retrieve the GID. The AID-GID pairing may also be changed or removed at any time by an Enrollment Authority exercising power at its discretion.

For clarification, the special terminologies used in the present invention must first be defined.

An Animal Blockchain Participant refers to any person or entity participating in the blockchain ecosystem by inputting onto or retrieving from the blockchain records of animals. They include owners who input their companion animals' data as well as veterinarians other pet service industry members who input onto or retrieve from the blockchain their client animals' data.

External storage (ES) refers to a separate data storage, which the animal blockchain may utilize to minimize overloading individual blocks on the blockchain as the whole of animal and human data may be too big to fit in a public block. Hence, only the essential data may be recorded as the transaction data in a block, with the bulk of the content being stored in an external data storage. An example of an external data storage is Decentralized Storage. The hash key, which is called ESID (External Storage ID) is included as a part of the transaction data in a block.

AID is a unique ID given to animals the time of enrollment onto the blockchain. AID may be in the form of a 128 bit UUID (universally unique identifier) according to the protocol of the RFC 4122 (ver. 4) standards, or any other uniquely generated identifier. The key advantage of using a scheme such as UUID is that it does not require any central server to ascertain its uniqueness.

GID is the owner or human ID, and is simply the public key used in the blockchain. For the sake of human readability and communication, an alias may be used.

Animal Registration Data (ARD) comprises all of the characteristic details of an animal (i.e. nickname, breed, color, birthday, etc.) as well as intrinsic biometric data (i.e. nose pattern image, nose template, full-body photo, face photo, and any distinguishing marks, etc.) While any other registration information, such as a government registration number or microchip information, may also be inputted, it should be noted that only biometric ID provides an animal with an ID that is intrinsic, tamper-proof and independent from the owner. Unlike AID, which is recorded as transaction data in a block in the blockchain at the time of enrollment, the whole of an animal's ARD may be quite big. ARD may be stored, in whole or in part, in the blockchain and/or an external storage. The hash key of an ARD is called ARD ESID (external storage ID). This ARD may be public data and freely accessible by the general public, or limited access to authorized third parties based on the terms of a constitution of the animal blockchain.

Guardian Additional Data (GAD) comprises the personal information of the owner. GAD may also be stored, in whole or in part, in the blockchain and/or an external storage with its hash key GAD ESID. In contrast to ARD, almost all of this GAD data is encrypted with the owner's public key so that only the owner has access. The only exception may be the contact information of the owner, which is needed as a means of getting in touch in case any such needs arise, like returning a lost companion animal to its owner.

Enrollment Authority refers to an entity that has the power to sign off on every enrollment transaction. At first glance, it may seem to be in the spirit of blockchain to allow anyone to unilaterally enroll one's companion animal without anyone else underwriting the action. However, if this were allowed, some thorny problems may emerge. For instance, if an owner dies or disappears without having transferred the ownership to another person, there is no way to officially assign another owner to that animal. The only way would be to enroll the animal under a different identity, which effectively defeats the whole purpose of having this blockchain as previous life data still would not be retrievable or linked to the new identity. Also, in cases of cruel treatment, some authoritative body should have the power to forcibly transfer ownership. For this reason, a special organization designated by the blockchain's constitution or elected by the blockchain's community with a good reputation, such as animal welfare NGOs, could be given the power to oversee the enrollment of animals. The Enrollment Authority may also exercise power at its discretion to make changes in guardianship.

Animal Records include records of enrollment as well as birth, death, medical adoption and other Life Data. At the core, the animal blockchain described is a public blockchain that records every data relevant to a companion animal's life. Said records are created and managed in the blockchain under the AID of the animal, which can be done by various Animal Blockchain Participants—which include persons or entities such as owners, veterinarians, service providers, rescue shelters, etc.—and linked to the AID to which the record pertains. Animal Records are accessible by the general public, or limited access to authorized third parties. It should be noted that the Enrollment Authority may play a role in verifying the authenticity of licenses of the various Animal Blockchain Participants, including veterinarians, granting them credentials as providers of genuine records. Life Data refers to the collection of events in the life of each specific companion animal and includes such matters as food purchases and employing grooming services and long-term and short-term caretaking services, etc. Each record may be signed with the appropriate person's signature, and a collection of such records is made into a block by a miner or block producer in the usual manner.

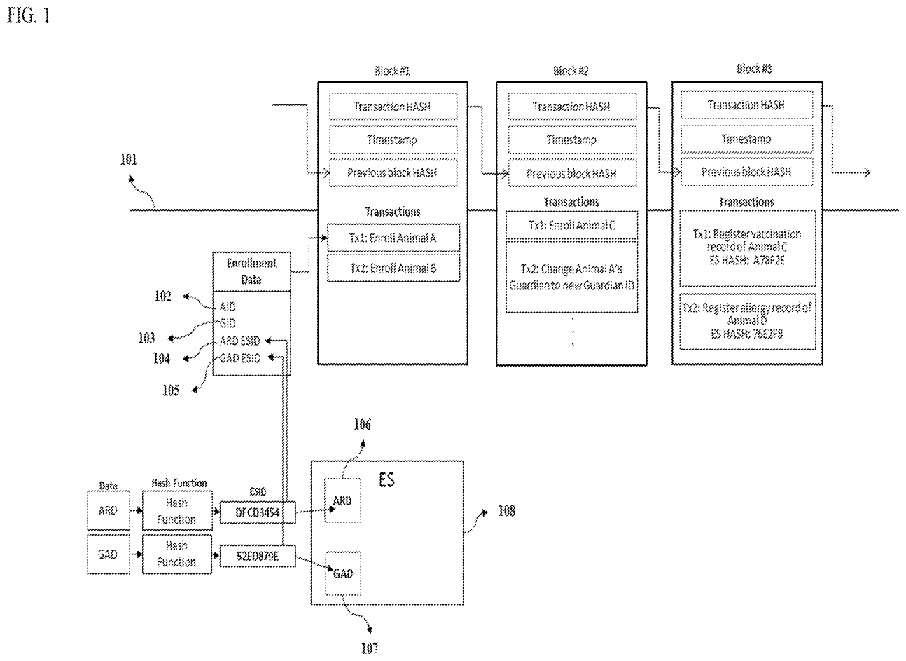

The most fundamental process that takes place in the blockchain is the enrollment of companion animal. FIG. 1 shows a typical enrollment process on the blockchain 101. In this embodiment, to enroll an animal, this set of four data items—AID 102, GID 103, ARD ESID 104, and GAD ESID 105—may to be recorded as the enrollment transaction in a block of the blockchain. Note that in this scenario ARD 106 and GAD 107 are stored on an external storage (ES) 108, with the enrollment transaction block only recording the hash keys (ESIDs) of their location on the external storage. It should also be noted that in this embodiment guardianship assignment is implicit in an enrollment transaction. When GID and GAD ESID are present, a specific guardian is assigned concurrently as the enrollment. However, in some cases, for example in the case of rescue animals without owners, enrollment may still occur but without GID and GAD ESID, wherein the enrollment transaction records only AID, ARD ESID, and null. In this case no specific guardian is assigned at the time of enrollment.

In practice, enrollment goes as follows: an animal's guardian (a permanent owner, or a temporary guardian like a rescue shelter personnel) sends the digitally signed enrollment application to the Enrollment Authority, who then digitally cosigns automatically and enrolls the animal.

Transferring ownership—including assigning new ownership to a previously assigned or unassigned animal, or removing an ownership without replacement follows a similar process. One simply repeats the whole enrollment and guardianship assignment process using a new owner ID or lack thereof (null). If there is no change in the ARD, the old ARD ESID can be used. From this blockchain's point of view, the same AID appears twice or maybe several times in scattered blocks, as many times as enrollment transactions have occurred for a particular animal. The simple rule is that for any particular animal, the most recent enrollment overrides all previous instances. Therefore, the correct current owner—or lack thereof—of an animal is the one that last appears with said animal on the blockchain in an enrollment transaction. Note that throughout this change of ownership process, only the AID-GID pairing is amended while all Animal Records remains unaffected. This easy decoupling mechanism of AID and GID is what makes this blockchain work as an animal-centric ledger.

Moreover, the linkages of AID, ARD, GID, and GAD enable the retrieval of one using another. One may retrieve the ARD of an animal using its AID. Or, the intrinsic biometric data of an animal recorded on the ARD may be used to retrieve the AID using biometric identification; wherein nose pattern data is used for nose pattern biometric recognition, and face or body data are used for facial or body recognition. Non-biometric registration data—such as government registration numbers or microchip information—on the ARD may also be used to retrieve the AID. The AID then may be used to retrieve the associated GID, which may then be used to retrieve the GAD.

An application of such a relationship would be locating the owner of a lost animal. When encountering a lost animal, the finder may try to identify the animal by submitting nose pattern images, face or full body photos to conduct an AID search for a match out of all the biometric data in the ARD stored in the blockchain or external storage. If a match is found, the finder can use the AID of the matched ARD to find the most recent enrollment transaction with a guardianship assignment to identify the GID associated with the assignment, and further obtain the contact information associated with said GID in the corresponding GAD.

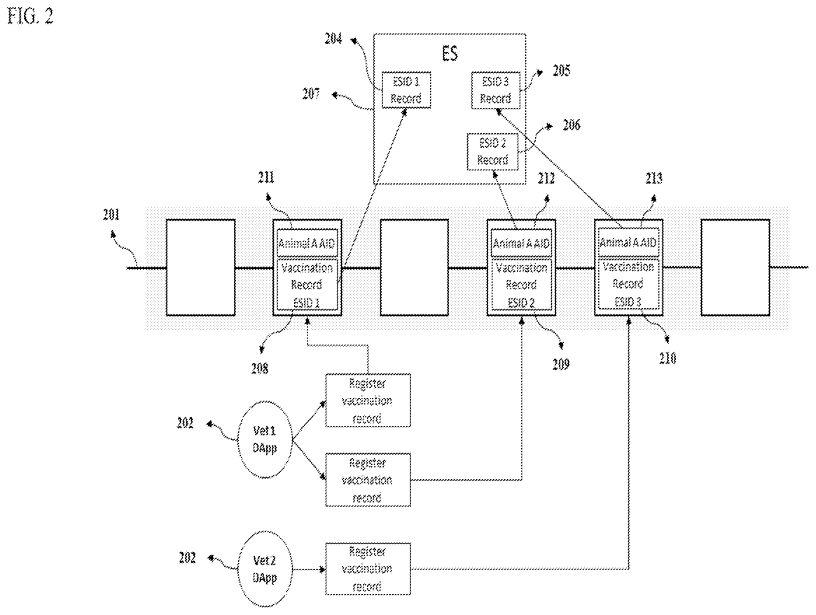

Of the many types of Animal Records, particularly important are medical records. Although vaccination is very important for the health of a companion animal and its surrounding community, keeping track of immunization records is no small task. It becomes even more problematic when an animal is lost or unexpectedly adopted by a new owner, in which case it is almost certain that the animal's old records are irreversibly lost. Keeping vaccination records on the blockchain alleviates this problem. FIG. 2 shows how vaccination records are kept in the blockchain. Each time an animal is vaccinated, the DApp (decentralized app) 101, 102 the veterinarian uses automatically signs the vaccination record 103, 104, 105 and loads it up to the blockchain. In fact, the vaccination detail may be stored in and external storage 106 and only its ESID 107, 108, 109 is recorded in a block in this blockchain, wherein a vaccination transaction data recorded in the blockchain is simply the pair of AID 110, 111, 112 and ESID of the vaccination detail stored in the external storage. Note that the record has nothing to do with an owner ID or data. This way, an animal's vaccination records are kept independent of any one owner or the change thereof. Moreover, once vaccination records are uploaded to the blockchain, there is no need for veterinarians to keep such records in their clinic either, and the comprehensive vaccination history of an animal can be made accessible at any time regardless of how many different veterinarians have administered them. When any need arises to collect all vaccination records to produce a vaccination certificate, for instance for foreign travel, a certificate issuing agency simply has to scan the blockchain by AID for vaccination transactions. One can then get hold of all vaccination ESIDs associated with the given AID.

The present invention may also use native tokens. The token has two different states: an active state for participating in various activities within the animal blockchain, and a tradable state for trading for another cryptocurrency or fiat currency (such as the US dollar). The owner of a token may convert the token state between tradable and active states at any time. Animal Blockchain Participants and block producers may be rewarded with tokens for certain activities.

Moreover, the present invention itself is a big data system on things related to animals. The health and medical data will be immensely useful for various future veterinary research, while other data may also be used to devise many innovative services such as customized and individualized food, care, and training services. The big data system may also allow for bulk data downloading for a cost as enumerated in the constitution of the blockchain. Furthermore, the bulk data downloading cost may be payable using the native tokens.

The components described in the example embodiments may be implemented by hardware components including, for example, at least one digital signal processor (DSP), a processor, a controller, an application-specific integrated circuit (ASIC), a programmable logic element, such as a field programmable gate array (FPGA), other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

Software may include a computer program, a piece of code, an instruction, or some combination thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. An animal blockchain comprising an enrollment of animals onto the blockchain using a unique Animal ID (AID).

2. The animal blockchain according to claim 1, wherein Animal Records are created and managed under the AID.

3. The animal blockchain according to claim 2, wherein the Animal Records are put on the blockchain.

4. The animal blockchain according to claim 3, wherein the Animal Records are accessible to authorized third parties.

5. The animal blockchain according to claim 4, wherein the authorized third party is a general public.

6. The animal blockchain according to claim 1, wherein the AID is linked to intrinsic biometric data of the animals.

7. The animal blockchain according to claim 6, wherein the AID is retrieved using the intrinsic biometric data.

8. The animal blockchain according to claim 1, wherein the AID is a universally unique identifier (UUID).

9. The animal blockchain according to claim 3, wherein the Animal Records are inputted by various Animal Blockchain Participants in the blockchain.

10. The animal blockchain according to claim 3, wherein the Animal Records are records of the enrollment or records of birth, death, medical, adoption, or other Life Data.

11. The animal blockchain according to claim 7, wherein the intrinsic biometric data is nose pattern data used for nose pattern biometric recognition.

12. The animal blockchain according to claim 7, wherein the intrinsic biometric data is used for at least one out of the group consisting of facial recognition and body recognition.

13. The animal blockchain according to claim 1, wherein AID is recorded as transaction data in a block in the blockchain and at least a part of an Animal Registration Data (ARD) is stored in the blockchain.

14. The animal blockchain according to claim 13, wherein remainder of the ARD is stored in an external storage and the reference of the external storage location is stored in the blockchain.

15. The animal blockchain according to claim 13, wherein the AID is retrieved using at least a part of the ARD.

16. The animal blockchain according to claim 1, wherein the enrollment comprises an animal's guardian sending a digitally signed enrollment application to an Enrollment Authority.

17. The animal blockchain according to claim 4, wherein an Enrollment Authority verifies the authenticity of licenses of the various Animal Blockchain Participants, including veterinarians, granting the Animal Blockchain Participants credentials as providers of genuine records.

18. The animal blockchain according to claim 1, wherein the animal blockchain comprises native tokens.

19. The animal blockchain according to claim 18, wherein the token comprises:
an active state for participating in various activities within the animal blockchain; and
a tradable state for trading for another cryptocurrency or fiat currency.

20. The animal blockchain according to claim 19, wherein an owner of the token converts the token state between the tradable and active states.

21. The animal blockchain according to claim 18, wherein blockchain participants are rewarded with the tokens.

22. The animal blockchain according to claim 18, wherein block producers are rewarded with the tokens.

23. The animal blockchain according to claim 3, wherein implementing the blockchain as a big data system comprises:
enumerating a cost for bulk data downloading in a constitution;
downloading the bulk data; and
paying the cost with native tokens.

24. An animal blockchain comprising a unique Animal ID (AID) and Guardian ID (GID) as paired entities.

25. The animal blockchain according to claim 24, wherein the AID and GID pairing changes during the animal's lifetime.

26. The animal blockchain according to claim 24, wherein the GID is retrieved using the AID.

27. The animal blockchain according to claim 25, wherein an Enrollment Authority exercises power at its discretion to make changes in the AID and GID pairing.
